(19) United States
(12) Patent Application Publication
(10) Pub. No.: US 2005/0090711 A1
(43) Pub. Date: Apr. 28, 2005

(54) System for locating lesions in hollow organs

(76) Inventors: Friedrich Fuchs, Roettenbach (DE); Rainer Kuth, Herzogenaurach (DE)

Correspondence Address: HARNESS, DICKEY & PIERCE, P.L.C., P.O.BOX 8910, RESTON, VA, 20195, US

(21) Appl. No.: 10/970,395

(22) Filed: Oct. 22, 2004

(30) Foreign Application Priority Data
Oct. 24, 2003 (DE) 10349659.9

(51) Int. Cl. A61B 1/04; A61B 1/06

(52) U.S. Cl. 600/113; 600/160; 600/178

(57) ABSTRACT

A system is for locating lesions in hollow organs. The system includes an endorobot with an integrated camera and integrated lighting device and with an RF transmitter and receiver unit with antenna. The system further includes an endorobot viewer unit with RF receiver unit and antenna. Further, a laparoscope is included, with an integrated camera and integrated lighting device and with insertable medical instruments. Finally, the system includes a laparoscope viewer unit.

The present application hereby claims priority under 35 U.S.C. §119 on German patent application number DE 103 49 659.9 filed Oct. 24, 2003, the entire contents of which are hereby incorporated herein by reference.

### FIELD OF THE INVENTION

The present invention relates generally to minimally invasive laparoscopy. The present invention relates particularly to a system and a method for improved determination of the position of lesions in hollow organs.

### BACKGROUND OF THE INVENTION

Hollow organs, including but not limited to the gastrointestinal tract (stomach and intestines), lungs (bronchi, bronchial branches), and brain (cranium), etc. are often the site of acute and/or chronic diseases. For example, this can be in the form of pathologically altered areas on the inside of an organ (tumors, polyps, etc.), hereinafter referred to as lesions, which over the course of time degenerate and become malignant. Epilepsy patients have areas of the brain where the signal fluctuations responsible for epilepsy seizures are initiated. Certain forms of cancer produce metastases in the brain, which lead to functional deficits.

The conventional therapeutic approach involves prompt removal, obliteration or other form of destruction of the lesions, for example by electrical or optical coagulation, by seed implantation, or injection of therapeutic agents. In doing this, a minimally invasive procedure, for example by way of an endoscope or laparoscope, would be desirable since such a procedure, compared to open surgery, allows the patient to recover more quickly. The problem, however, is that many disease sites and many disease types inside the hollow organs can be reached by endoscope or laparoscope only with great difficulty, if at all.

For example, it is not possible to view the entire length of the intestine (up to 11 meters) by endoscopy, despite the fact that many inflammatory or neoplastic processes are situated in the middle region of the intestine. Furthermore, laparoscopic detection and diagnosis of lesions (i.e. by access from the outside) is often not possible because the lesions lie hidden in the organ wall and are therefore not visible from the outside and/or they are concealed by other organs or organ parts.

### SUMMARY OF THE INVENTION

It is therefore an object of an embodiment of the present invention to make available a system and a method with which diseases or lesions of hollow organs can be at least one of located, diagnosed and treated in a minimally invasive manner.

According to an embodiment of the invention, therefore, a system for locating lesions in hollow organs includes,
an endorobot with an integrated camera and integrated lighting device and with an RF transmitter and receiver unit with antenna,
an endorobot viewer unit with RF receiver unit and antenna,
a laparoscope with an integrated camera and integrated lighting device and with insertable medical instruments, and
a laparoscope viewer unit.

The system advantageously may have an endorobot control apparatus. The system may also advantageously have an endorobot navigation system. Moreover, the system advantageously may have a laparoscope navigation system.

According to an embodiment of the invention, the lighting device of the endorobot and/or of the laparoscope may be composed of a high-intensity pulsed light source. Advantageously, the light of the lighting device of the endorobot and/or of the laparoscope can be chosen spectrally. Advantageously, the laparoscope may be composed exclusively of non-ferromagnetic parts.

For user-friendly operation of the system, according to an embodiment of the invention, the endorobot control apparatus may include a laparoscopic access.

The endorobot viewer unit also advantageously may have an RF transmitter unit for issuing commands to the endorobot and/or for charging an energy store of the endorobot.

Moreover, according to an embodiment of the invention, a method for locating and diagnosing lesions in hollow organs includes:
introducing an endorobot into the interior of a hollow organ to be examined,
navigating the endorobot in the interior of the hollow organ via an endorobot control apparatus until a lesion is found,
bringing a laparoscope to the position of the lesion from outside the hollow organ to be examined, via an opening made in the body in a minimally invasive surgical procedure,
diagnosing the lesion through interaction of endorobot and laparoscope.

In a possible advantageous embodiment of the present invention, the position of the lesion may be determined via an endorobot navigation system. In a further embodiment, the position of the lesion may be determined by way of detection of light signals from the endorobot by the laparoscope.

A further determination of the position of the lesion may be done by X-ray, ultrasound or MRT measurements.

According to an embodiment of the invention, the diagnosis takes place in the form of fluoroscopy and/or contrast-assisted illumination by means of endorobot and/or laparoscope.

According to an embodiment of the invention, medical treatment of the lesion is advantageously done via laparoscope and/or endorobot or by a combination of both, by electrical or optical coagulation, biopsy, seed implantation, or injection of therapeutic agents.

It should be noted that, according to an embodiment of the invention, a laparoscope may be used which is not ferromagnetic.

The method according to an embodiment of the invention may be advantageously performed on the gastrointestinal tract, the lungs, the cranium, and the amniotic sac.

According to an embodiment of the invention, commands may be issued to the endorobot and/or an energy store of the endorobot may be charged via an RF transmitter unit at the endorobot viewer unit.

Furthermore, a medical procedure may include one, according to an embodiment of the invention, in which the physician, with visual monitoring via an endorobot, introduces instruments minimally invasively into a hollow organ to be diagnosed and treated and carries out differentiated diagnosis and/or treatment in a minimally invasive manner.

The medical procedure may further include the minimally invasive instrument being introduced at a location of the hollow organ which appears suitable from the visual monitoring via the endorobot.

### BRIEF DESCRIPTION OF THE DRAWINGS

Further advantages, features and characteristics of the present invention are now explained in more detail on the basis of illustrative exemplary embodiments and with reference to the attached drawings, which are given by way of illustration only and thus are not limitative of the present invention, and wherein.

FIG. 1 is a diagrammatic representation of the system according to an embodiment of the invention for locating lesions in hollow organs, using the example of an intestinal polyp and on the basis of the combination, according to an embodiment of the invention, of endorobot and laparoscope.

FIG. 2 shows, in particular, the opto-electronic components integrated in the endorobot, and the viewer devices for viewing the images from the endorobot and the images from the laparoscope.

### DETAILED DESCRIPTION OF THE EXEMPLARY EMBODIMENTS

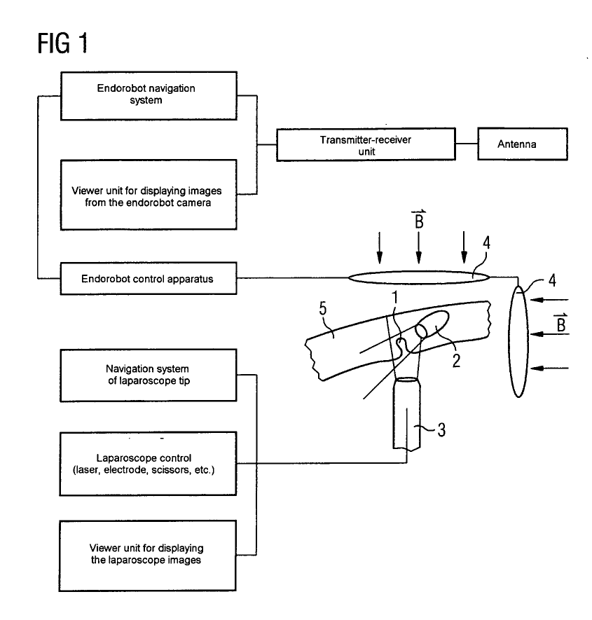

FIG. 1 is a diagrammatic and very much simplified representation of the system according to an embodiment of the invention which makes it possible to locate lesions in hollow organs that are difficult to access by endoscopy, and which also permits their subsequent diagnosis and treatment by the user. The system and the way it functions are explained using the example of a polyp in the small intestine.

The figure indicates a portion of the small intestine 5 which, at a site not easily accessible and not visible from outside (by laparoscopy), has a lesion in the form of a polyp 1. The system according to an embodiment of the invention allows the user to find the polyp. For this purpose, an endorobot is introduced into the gastrointestinal tract.

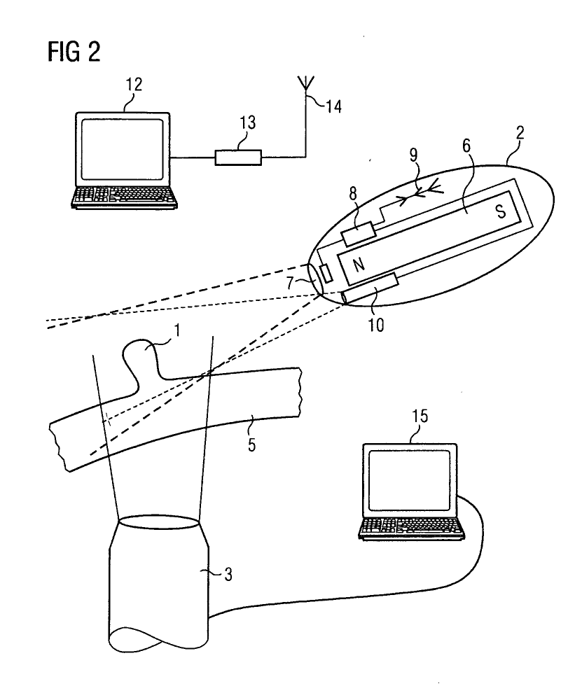

The opto-electronic equipment of the endorobot (camera, lamp, bar magnet, RF transmitter-receiver unit, antenna, etc.) is shown in detail in FIG. 2 and allows the user, in combination with an endorobot control apparatus and on the basis of transmitted image data, to navigate the endorobot through the intestine until the camera 7 of the endorobot has optically detected the polyp illuminated by the lamp 10. For this purpose, anatomical images of the intestine are transmitted continuously (at a frequency of 2 Hz) from the endorobot camera 7, via the endorobot transmitter and receiver unit 8 and via the endorobot antenna 9, to the antenna 14 and the RF receiver unit 13 of the endorobot viewer unit 12 and are converted to high resolution and optionally displayed on the latter. If appropriate, the endorobot viewer unit 12 also has an RF transmitter unit via which commands can be issued to the endorobot 2 or via which an energy store of the endorobot can be charged.

The navigation is performed by the user via a power input appliance (for example a so-called 6D mouse) through which the magnetic fields \vec{B} from the gradient coils surrounding the patient are accordingly varied and, in this way, a torque and/or a translatory force is applied to the bar magnet in the inside of the endorobot. For the sake of clarity, only two gradient coils are shown in FIG. 1.

An endorobot control apparatus for controlling an endorobot in magnetic field control systems is set out in detail in patent specification DE 101 42 253 C1, the entire contents of which are hereby incorporated herein by reference. An endorobot which has the integrated components mentioned is described in patent specification US 6,240 312 B1, the entire contents of which are hereby incorporated herein by reference.

The endorobot now has several tasks. On the one hand, after optical detection of the polyp, its position corresponds substantially to the position of the polyp and therefore serves for targeted introduction of a laparoscope. The actual determination of the endorobot position can be effected in different ways.

If the endorobot control apparatus or the transmitter-receiver unit is connected to an endorobot navigation system, the endorobot position can be displayed in a simple manner (for example on the viewer unit) and read off. If no endorobot navigation system is present, the endorobot position can be inferred from X-ray, ultrasound, CT or MRT measurements.

Another possibility is to have the endorobot emit optical signals (for example flashes by laser pulsed operation) which can be detected by an optical sensor at the laparoscopy tip. When the position of the endorobot and therefore that of the polyp has been determined, the tip of a laparoscope can be brought (for example with the aid of a navigation system of the laparoscope) to the diseased site of the intestinal wall, and a laparoscopic access can thus be made to the polyp which is to be diagnosed and treated. The laparoscopic access permits, on the one hand, insertion of a large number of laparoscopic instruments needed for the diagnosis and treatment, and, on the other hand, further display of the anatomy on a further viewer unit 15 of the laparoscope.

For a differentiated diagnosis, the endorobot has the further task of illuminating the polyp and the inside of the intestinal wall especially brightly or spectrally so that it is possible by way of laparoscopy to examine the organ wall by transmitted light or, conversely, to detect especially bright or spectral lighting of the tissue through the laparoscope and display this on the endorobot viewer unit. In particular a spectral illumination (for example in the red or UV range) in combination with contrast agents, which have fluorescing properties in the corresponding frequency range, permit differentiated diagnosis in respect of the malignancy and condition of the pathological tissue.

An especially bright lighting both of the endorobot and of the laparoscope is advantageously effected by pulsed light sources, for example by way of xenon flash lamps, LEDs in pulsed mode, or pulsed lasers. It should be noted here that, by application of extremely high luminous power, heating and consequent damage of the tissue is avoided.

Furthermore, the endorobot has the diagnostic task of showing the user the vascular state of the organ in the diseased area, in order to help avoid serious bleeding during a therapeutic intervention. Tumors especially, through an enzymatic action on the surrounding tissue, are able to intensify the formation of blood vessels, which in the final analysis provide a better supply to the tumors themselves. Moreover, the images from the endorobot can be used to evaluate the intestinal wall in order to establish the laparoscopic access where the least possible damage occurs. Additional damage to already diseased tissue in the context of a therapeutic intervention generally entails a longer period of healing.

Because of its small size (up to ca. 1 cm) and limited load, the endorobot is restricted in terms of the part it can play in the intervention, for which reason the actual treatment takes place in a substantially minimally invasive manner via suitable instruments of the laparoscope. According to an embodiment of the invention, however, a treatment procedure takes place at all times under visual monitoring via the endorobot, for which reason the endorobot also has to be navigated during the operation.

The use of a laparoscope in combination with an endorobot control apparatus, which surrounds the patient on all sides with coils, requires an (upper) opening which permits use of a laparoscope. It is also necessary to use a laparoscope and laparoscopic instruments which are made of non-ferromagnetic materials, in order to avoid bending and twisting during surgical use in the presence of (changing) magnetic fields.

The example shown in FIG. 1 can be extended to other hollow organs. As has already been mentioned in the introductory part of the description, the combination, according to an embodiment of the invention, of an endorobot and a laparoscope is practicable in the case of (tumorous) diseases in the brain, in the bronchial branches of the lungs, and also in the amniotic sac, in other words in all situations where the use of an endorobot means that the subsequent laparoscopic intervention is made easier and optimized. In particular, three-dimensional objects, which are recorded two-dimensionally by way of endorobot and/or laparoscopy lens, can, by suitable image processing techniques, be represented three-dimensionally and thus make diagnosis easier for the user.

Exemplary embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A system for locating lesions in hollow organs, comprising:
an endorobot, including an integrated camera and integrated lighting device and including an RF transmitter and receiver unit with antenna;
an endorobot viewer unit with RF receiver unit and antenna;
a laparoscope with an integrated camera and integrated lighting device and with insertable medical instruments; and
a laparoscope viewer unit.

2. The system as claimed in claim 1, further comprising an endorobot control apparatus.

3. The system as claimed in claim 1, further comprising an endorobot navigation system.

4. The system as claimed in claim 1, further comprising a laparoscope navigation system.

5. The system as claimed in claim 1, wherein the lighting device of at least one of the endorobot and the laparoscope includes a high-intensity pulsed light source.

6. The system as claimed in claim 1, wherein the light of the lighting device of at least one of the endorobot and the laparoscope is chosen spectrally.

7. The system as claimed in claim 1, wherein the laparoscope is composed exclusively of non-ferromagnetic parts.

8. The system as claimed in claim 2, wherein the endorobot control apparatus includes a laparoscopic access.

9. The system as claimed in claim 1, wherein the endorobot viewer unit includes an RF transmitter unit for at least one of issuing commands to the endorobot and for charging an energy store of the endorobot.

10. A method for locating and diagnosing lesions in hollow organs, comprising:
introducing an endorobot into the interior of a hollow organ to be examined;
navigating the endorobot in the interior of the hollow organ via an endorobot control apparatus until a lesion is found;
bringing a laparoscope to the position of the lesion from outside the hollow organ to be examined, via an opening made in the body in the context of a minimally invasive surgical procedure; and
diagnosing the lesion through interaction of endorobot and laparoscope.

11. The method as claimed in claim 10, wherein the position of the lesion is determined via an endorobot navigation system.

12. The method as claimed in claim 10, wherein the position of the lesion is determined via detection of light signals from the endorobot by the laparoscope.

13. The method as claimed in claim 10, wherein the position of the lesion is determined by at least one of X-ray, ultrasound and MRT measurements.

14. The method as claimed in claim 10, wherein the diagnosis takes place in the form of at least one of fluoroscopy and contrast-assisted illumination by use of at least one of the endorobot and laparoscope.

15. The method as claimed in claim 10, further comprising treating the lesion, using at least one of the laparoscope and the endorobot, by at least one of electrical or optical coagulation, biopsy, seed implantation, and injection of therapeutic agents.

16. The method as claimed in claim 10, wherein a laparoscope is used which is not ferromagnetic.

17. The method as claimed in claim 10, wherein the method is performed on at least on of the gastrointestinal tract, the lungs, the cranium, and the amniotic sac.

18. The method as claimed in claim 10, wherein commands are issued to the endorobot via an RF transmitter unit at the endorobot viewer unit.

19. The method as claimed in claim 10, wherein an energy store of the endorobot is charged by an RF transmitter unit at the endorobot viewer unit.

20. A medical procedure, comprising:
introducing, with visual monitoring via an endorobot, minimally invasive instruments into a hollow organ to be at least one of diagnosed and treated; and
carrying out at least one of differentiated diagnosis and treatment in a minimally invasive manner.

21. The medical procedure as claimed in claim 20, wherein the minimally invasive instrument is introduced at a location of the hollow organ which appears suitable from the visual monitoring via the endorobot.

22. The system as claimed in claim 2, further comprising an endorobot navigation system.

23. The system as claimed in claim 2, further comprising a laparoscope navigation system.

24. The system as claimed in claim 3, further comprising a laparoscope navigation system.

25. The method as claimed in claim 15, wherein the method is performed on at least on of the gastrointestinal tract, the lungs, the cranium, and the amniotic sac.
